(19) United States
(12) Patent Application Publication
(10) Pub. No.: US 2022/0117333 A1
(43) Pub. Date: Apr. 21, 2022

(54) HEAD GARMENT WITH MULTI-LAYER FILTER

(71) Applicants: Janet McCreesh, Flushing, NY (US); Diane Neligan Sabena, Flushing, NY (US)

(72) Inventors: Janet McCreesh, Flushing, NY (US); Diane Neligan Sabena, Flushing, NY (US)

(21) Appl. No.: 17/072,666

(22) Filed: Oct. 16, 2020

(51) Int. Cl. A41D 13/11; A42B 1/04; B32B 5/26; A42B 1/24; A42B 1/22; A62B 18/02; A62B 23/02

(52) U.S. Cl. CPC A41D 13/1161; A42B 1/046; B32B 5/26; A41D 13/1184; B32B 2571/00; A42B 1/22; A42B 1/248; A62B 18/02; A62B 23/02; A42B 1/247

(57) ABSTRACT

A head garment is made of elastically stretchable fabric configured to fit over and surround the head of a wearer and conform substantially to the shape of the wearer's head. The head garment has an eye opening positioned to allow the wearer to see therethrough, and a protective eye shield completely covering the eye opening. A multi-layer filter portion is configured to cover the wearer's mouth and at least a lower part of the wearer's nose and has three or more filter layers for filtering air during breathing. Additional filter layers may be added to the multi-layer filter portion to enhance or modify the filtering properties. The multi-layer filter portion may be configured as a removable filter pack that can be removed and replaced by other filter packs having different filtering properties.

### FIELD OF THE DISCLOSURE

The present disclosure relates generally to head garments configured to fit over and surround the head of a person to provide protection from the surrounding environment, and in particular, to such head garments which have a multi-layer filter for filtering air during breathing.

### BACKGROUND OF THE DISCLOSURE

Filter masks are commonly worn over the nose and mouth of a person to prevent contaminants and other substances from entering the wearer's respiratory system and/or to protect other persons or things from being exposed to pathogens and other contaminants exhaled by the wearer.

In recent years, there has been an increased awareness and concern for preventing contamination and infection of the public and health care personnel by airborne pathogens. Therefore it has become necessary to prevent the spread of infections from person to person and from patient to health care worker and vice versa by preventing inhalation or exhalation of airborne infectious aerosols and other particles. More recently, the outbreak of SARS-CoV-2, commonly known as the novel coronavirus, has become even a greater concern due to the fact that the viral particles spread easily and quickly. When people infected with the virus breathe out, clear their throats, cough, sneeze, speak or otherwise expel air out their nose or mouth, droplets of different sizes, which contain the virus, are ejected into the air.

Numerous face masks have been developed to help prevent people infected with the coronavirus from spreading the virus. However, such face masks generally cover only the nose and mouth of the wearer and do not provide protection for other parts of the wearer's head or neck. For example, a face mask does not protect the wearer's face and neck from ultraviolet (UV) light or protect the wearer's head from annoying, and sometimes dangerous, insects such as mosquitoes, flies, gnats, wasps and bees. Wearers of face masks have difficulty maintaining the masks snugly against their faces, resulting in leakage of air and lowering of filter performance. Additionally, conventional face masks are not made or intended to be worn wet and are of limited use to people participating in water activities at oceans, lakes, rivers and pools where people, particularly children, are often in close contact.

### SUMMARY OF THE DISCLOSURE

The present disclosure concerns head garments made of elastically stretchable fabric configured to fit over and surround the head of the wearer and conform substantially to the shape of the wearer's head. The head garment has an eye opening positioned to allow the wearer to see therethrough, and a multi-layer filter portion configured to cover the wearer's mouth and at least a lower part of the wearer's nose for filtering air during breathing. The head garment may be made in various sizes to stretch a slight extent and fit, without discomfort, over the heads of toddlers, preschoolers and other children and in larger sizes for adults.

One aspect of the head garment includes a protective eye shield permanently and non-removably attached to the head garment. The protective eye shield is transparent and may have one or more of scratch-resistant, fogging-resistant, and ultraviolet (UV) light-blocking properties.

Another aspect of the head garment includes a protective eye shield detachably attached to the head garment, which enables the protective eye shield to be removed when the head garment is washed. The protective eye shield also may be removed and replaced by another one in the event the eye shield becomes scratched or otherwise damaged.

In one example, the protective eye shield comprises goggles which removably fit over the head garment while worn by the wearer. In another example, the protective eye shield comprises a transparent member fixed to a flexible frame. The flexible frame is detachably attached to the head garment around the eye opening by fasteners. The fasteners have first mating parts fixed to the head garment in spaced relation around the eye opening and second mating parts fixed in spaced relation to the underside of the flexible frame. The first mating parts align with corresponding second mating parts when the flexible frame is aligned with the eye opening thereby enabling quick and easy attachment and detachment of the protective eye shield.

A further aspect of the head garment includes a multi-layer filter portion comprised of three or more filter layers superposed over one another. One filter layer is the fabric of the head garment, which is the outer filter layer, and two filter layers are fixed to the backside of the outer filter layer and constitute middle and inner filter layers. The middle and inner filter layers are attached along their sides and bottoms to the backside of the outer filter layer and are not attached along their tops thereby creating an opening between the outer and middle filter layers and an opening between the middle and inner filter layers. The two openings open into respective compartments, one between the outer and inner filter layers and the other between the middle and inner filter layers. Additional filter layers may be inserted from the inside of the head garment (when the head garment is not being worn) into the compartments to enhance or modify the filtering properties of the multi-layer filter portion.

Another aspect of the head garment includes a removable multi-layer filter portion which is removably attached to the inside of the front face of the head garment. This enables the filter portion to be removed for cleaning and sterilization or for exchange with another filter portion. An assortment of different filter portions may be provided with filter layers of different compositions and/or different numbers of filter layers.

A further aspect of the head garment includes configuring the head garment as a fictitious character, such as the head of an animal, superhero and the like, which would appeal to children and encourage them to wear the head garment.

Other aspects and advantages of this disclosure will become apparent upon reading the following description in conjunction with the accompanying drawings.

### BRIEF DESCRIPTION OF THE DRAWINGS

Embodiments of the head garment are illustrated by way of example in the accompanying drawings, in which like reference numerals refer to like parts in the different embodiments.

FIG. 1 is a front view of one embodiment of a head garment illustrating the configuration of the head garment when worn by a user and showing the outer layer of the multi-layer nose-and-mouth filter portion.

FIG. 2 is a rear view of the head garment illustrated in FIG. 1.

FIG. 3 is a front view of another embodiment of a head garment illustrating the head garment worn by a user and showing the nose-and-mouth filter portion partially cut and folded back to illustrate the multiple filter layers.

FIG. 4 is a front view of one embodiment of a protective eye shield detachably attached to the head garment.

FIG. 5 is a front view of another embodiment of a protective eye shield detachably attached to the head garment.

FIG. 6 is a view of the backside of the front of the head garment illustrated in FIG. 1, as seen from inside the head garment, showing the multiple filter layers of the nose-and-mouth filter portion.

FIG. 7 is an enlarged view of a section of the nose-and-mouth filter portion looking downward along line 7-7 in FIG. 6.

FIG. 8 is a front view of a further embodiment of a head garment worn by a user, the head garment having a removable nose-and-mouth filter portion which is partially cut and folded back to illustrate the multiple filter layers.

FIG. 9 is a front view of another embodiment of a head garment worn by a user, the head garment being configured as a fictitious character.

### DETAILED DESCRIPTION

The present disclosure relates to head garments made of elastically stretchable fabric configured to fit over and surround the head of the wearer and conform substantially to the shape of the wearer's head. The head garment has an eye opening positioned to allow the wearer to see therethrough, and a multi-layer filter portion configured to cover the wearer's mouth and at least a lower part of the wearer's nose for filtering air during breathing. The multi-layer filter portion has three or more filter layers and allows the wearer to breathe without restriction. Various aspects of the head garment are described in the following description with reference to different embodiments and examples, which illustrate exemplary ways of implementing the various aspects.

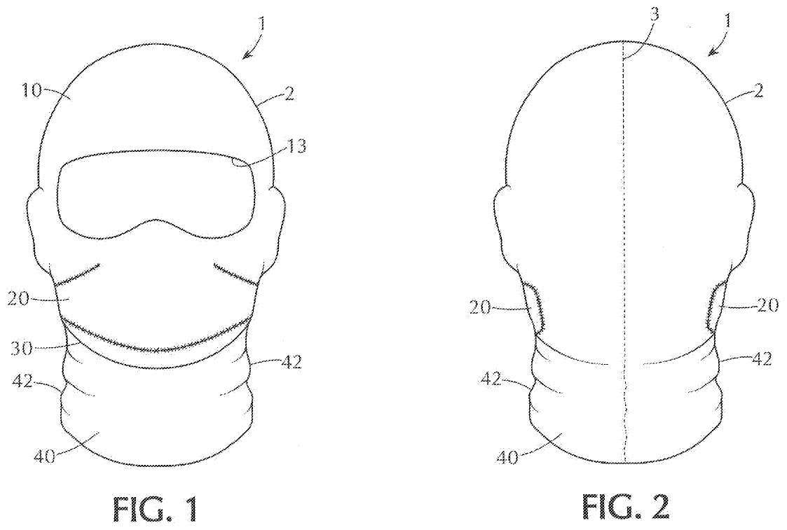

FIGS. 1-2 illustrate one embodiment of a head garment 1 configured to fit over and surround the head and neck of a wearer. The head garment is comprised of elastically stretchable fabric 2 having a suitable level of elasticity that enables the garment to stretch and fit with a mild degree of compression over the head of the wearer and conform to the shape of the wearer's head. When worn, the fabric 2 stretches to accommodate the wearer's nose, ears and chin, which helps hold the head garment 1 in place without the need of straps, snaps, ties or other securing means. The head garment 1 may be made in various sizes to stretch to varying degrees where necessary to fit, with minimal discomfort, over the heads of toddlers, preschoolers and other children and in larger sizes for adults.

The elastically stretchable fabric 2 may be a blend of spandex and nylon or polyester such as commonly used in rash guards. These fabrics have many desirable properties which make them highly suitable for use in manufacturing the head garments, notably the properties of strength, elasticity, antibacterial, UV protection, breathability, light weight, water wicking, chafing and rash protection, resistance to perspiration and body oils, and washability.

In the embodiment of FIGS. 1-2, the head garment 1 is cut, using a pattern, from one piece of fabric and sewn together along a seam 3 at the rear of the garment. Alternatively, the head garment may be manufactured from multiple fabric pieces which are sewn together to form the garment. Flatlock seams are preferred so that the seams will lie flat against the wearer thereby providing greater comfort. Other appropriate flat seam constructions may be used instead of sewing, such as heat bonding or ultrasonic welding, where the seams are butted or joined in an over-lapped orientation.

The head garment 1 has an upper portion 10 configured to fit over and surround the upper part of a wearer's head including the eyes, ears and upper part of the nose. The upper portion 10 is provided with an eye opening 13 positioned to be in front of the eyes of a user wearing the garment. The eye opening 13 extends completely through the fabric 2 and is configured to provide the user an unencumbered, substantially full range of vision. In the illustrated embodiment, the eye opening 13 is a single opening. Alternatively two separate eye openings may be provided and positioned to be in front of respective ones of the wearer's two eyes.

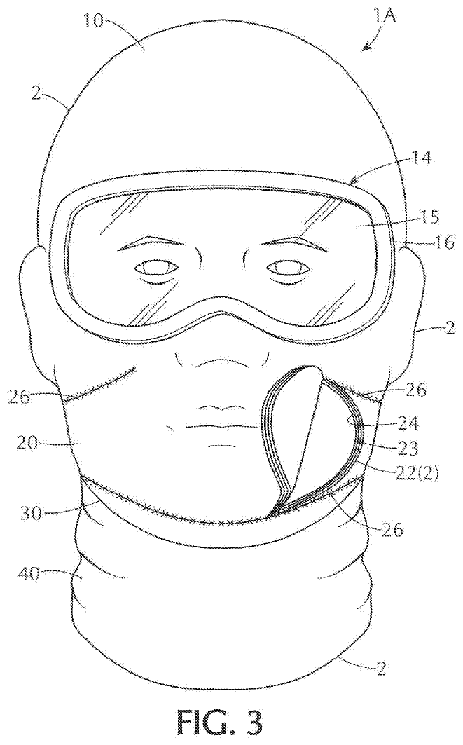

A nose-and-mouth filter portion 20 (sometimes referred to hereinafter as filter portion 20) extends downward from the upper portion 10 and is configured to cover at least the lower part of the nose and the mouth of a wearer wearing the garment. The filter portion 20 filters the air inhaled and exhaled by the wearer and is described below with reference to FIGS. 3, 6 and 7. The lower side of the filter portion 20 preferably extends above the wear's chin. A chin portion 30 of the garment 10 extends downward from the filter portion 20 to cover the chin of the wearer and aids in holding the garment in place. The chin portion 30 terminates in a neck portion 40 configured to fit loosely around the neck of the wearer. The loose fitting neck portion gathers together and forms loose folds 42 to prevent the head garment 1 from exerting compression on the wearer's neck.

The filter portion 20 is integral with the upper portion 10 and the chin portion 30 and fits snugly over the wearer's nose and mouth thereby preventing air leakage around the filter portion. This is an advantage over conventional filter masks in which air often leaks between the filter mask and the wearer's face, especially if the filter mask is not maintained snugly against the face of the wearer.

FIG. 3 illustrates another embodiment of a head garment 1A which is similar to the FIGS. 1-2 embodiment except includes a protective eye shield 14 covering the eye opening 13. FIG. 3 illustrates the garment 1A worn by a wearer and shows the nose-and-mouth filter portion 20 partially cut and folded back to show the multiple filter layers. The protective eye shield 14 comprises a transparent member 15 fixed to a flexible frame 16 made of plastic, rubber or other suitable material that has sufficient flexibility to fit snugly against the wearer's face.

The transparent member 15 may be made of PETG (polyethylene terephthalate glycol) or polycarbonate, both of which are strong and scratch resistant, durable and have excellent optical clarity, or may be made of other clear plastics having similar properties. The transparent member 15 may be tinted or polarized to provide protection from sunlight and ultraviolet (UV) light. The transparent member 15 is fixed by adhesive, snap fit or other suitable fixation to the flexible frame 16. The flexible frame 16 is securely adhered, such as by heat bonding, ultrasonic welding or suitable bonding agent, on its rear side to the fabric 2 around the marginal edge portion of the eye opening 13. In this embodiment, the eye shield 14 is permanently and non-removably attached to the head garment.

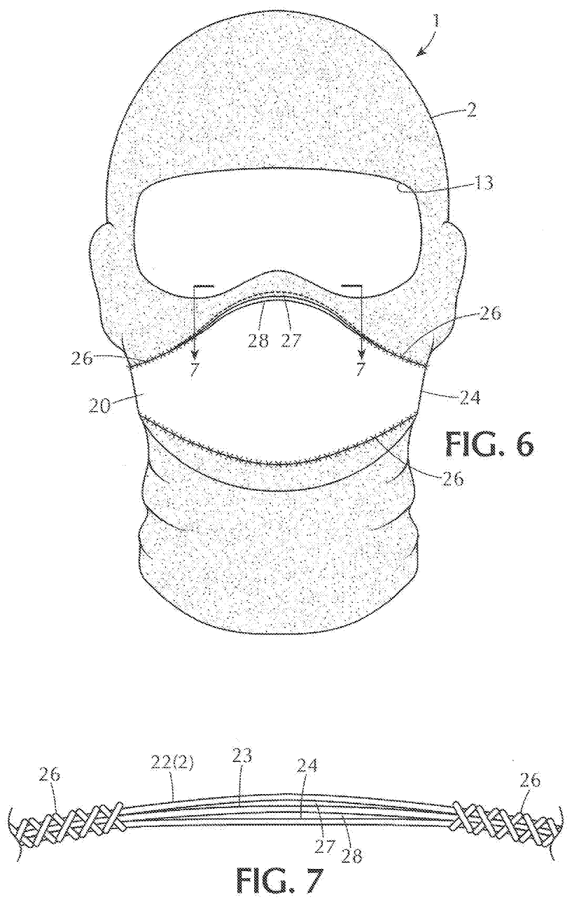

The nose-and-mouth filter portion 20, in this example, has a three-layer construction comprised of an outer filter layer 22, which is a region of the fabric 2, a middle filter layer 23 and an inner filter layer 24. The middle and inner filter layers 23, 24 are attached to the backside of the outer filter layer 22 by stitching 26 as described in more detail below with reference to FIGS. 6-7. Instead of stitching, the middle and inner filter layers 23, 24 may be attached to the outer filter layer 22 by heat bonding, ultrasonic welding or other means. During manufacture, and before the cut patterned fabric 2 is sewn or otherwise joined together to form the head garment, the middle and inner filter layers 23, 24 are superposed on the backside of the outer filter layer 22, i.e., the backside of the fabric 2, and the middle and inner filter layers are jointly sewn to the outer filter layer along the stitch line 26 thereby forming the three-layer filter portion 20. Though one stitch line 26 is shown, two or more stitch lines may be used for a more secure attachment.

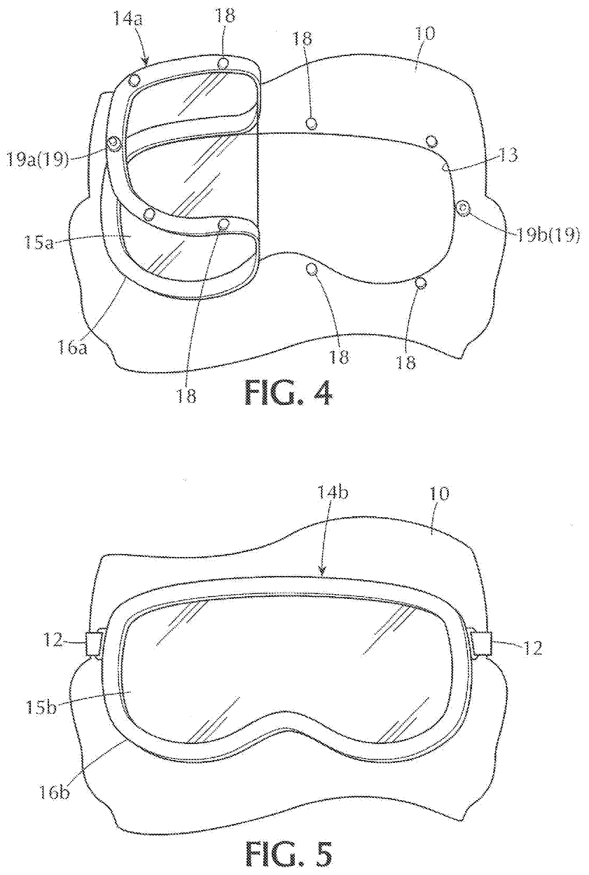

FIG. 4 illustrates one embodiment of a protective eye shield 14a that is detachably attached to the upper portion 10 of the head garment to cover the eye opening 13. The protective eye shield 14a comprises a transparent member 15a fixed to a flexible frame 16a. The transparent member 15a may be made of PETG, polycarbonate, or other suitable clear plastics and may be tinted or polarized to provide protection from sunlight and ultraviolet (UV) light. The flexible frame 16a may be made of rubber, plastic or other suitable material having sufficient flexibility to fit snugly against the wearer's face. The protective eye shield 14a is detachably connected to the head garment by fasteners. The detachability of the protective eye shield 14a is advantageous and allows for washing of the head garment with the protective eye shield removed thereby avoiding damaging the protective eye shield. Other advantages are a damaged protective eye shield may be removed and repaired or replaced by a new one, and a protective eye shield without sunlight protection, for example, may be replaced by one having a transparent member tinted and/or polarized to prevent bright sunlight and UV light from damaging or discomforting the wearer's eyes.

In this embodiment, the fasteners comprise disc magnets 18, snaps 19, or a combination of disc magnets and snaps. Other suitable fasteners may be used. Each fastener has two mating parts, one part fixed to the marginal edge portion of the fabric 2 around the periphery of the eye opening 13 and the other part fixed to the backside of the frame 16a. The disc magnet fasteners each comprise two mating disc magnets 18, and the snap fasteners 19 each comprise mating male and female parts 19a and 19b, the male part 19a having a protrusion that snap fits in an opening in the mating female part 19b. The mating parts of the fasteners on the marginal edge portion of the fabric 2 are positioned to align with the corresponding mating parts of the fasteners on the frame 16a when the protective eye shield 14 is aligned with the eye opening 13 thereby enabling quick, simple and accurate attachment of the protective eye shield to the head garment and easy detachment of the protective eye shield from the head garment.

Another embodiment of a protective eye shield 14b that is detachably attached to the upper portion 10 of the head garment to cover the eye opening 13 is shown in FIG. 5. In this embodiment, the protective eye shield 14b comprises goggles having a transparent member 15b fixed by adhesive, snap fit or other suitable fixation to a flexible frame 16b. The transparent member 15b may be made of PETG, polycarbonate, or other suitable clear plastics and may be tinted or polarized to provide protection from sunlight and ultraviolet (UV) light. The flexible frame 16b is made of rubber, plastic or other suitable material having sufficient flexibility to fit the goggles 14b snugly against the wearer's face. An elastic strap 12 is connected to opposite ends of the frame 16b. The length of the elastic strap 12 that extends between the opposite ends of the frame 16b is adjustable and, prior to donning the head garment, the length of the elastic strap 12 is adjusted in conjunction with the flexibility of the frame 16b to maintain the goggles snugly against the wearer's face.

The nose-and-mouth filter portion 20 shown in FIGS. 1-3 is next described with reference to FIGS. 6-7. FIG. 6 is a view of the backside of the front of the head garment 1 illustrated in FIG. 1, i.e., the backside of the fabric 2 viewed from inside the head garment. The backside of the fabric 2 is stippled to differentiate it from the front side. FIG. 7 is an enlarged view of a section of the filter portion 20 looking downward along line 7-7 in FIG. 6. The three-layer filter portion 20 comprises the outer filter layer 22, which is the fabric layer 2 and is denoted by a dashed line in FIG. 6, the middle filter layer 23 and the inner filter layer 24. The middle and inner filter layers 23, 24 have substantially the same shape and size. The inner filter layer 24 is superposed on the middle filter layer 23, and the two filter layers are superposed on and attached to the outer filter layer 22 (the backside of the fabric layer 2) by stitching 26 or by heat bonding, ultrasonic welding or other means.

As used herein, “filter layer” means a layer of fabric (natural or synthetic) or other material which has the property of blocking or removing unwanted particles from air passing through the filter layer. The filter layers of the filter portion 20 block or remove particles in the air inhaled by the wearer, preventing the particles from entering the wearer's respiratory system, and block or remove particles in the air exhaled by the wearer, preventing other persons or things from being exposed to the particles. By way of example, the filter layers may be fabric layers made of cotton, natural silk, spandex, polyester, polypropylene, nylon, chiffon made of thermoplastic fibers, and the like. Filter layers made of natural silk, polyester chiffon or other materials have electrostatic properties and provide an electrostatic barrier against droplets. The tightness of the weave is important, and fabrics having higher thread counts have higher filter efficiencies than the same fabrics having lower thread counts. Multi-layer filters having a combination of ne filter layer exhibiting electrostatic properties and one or more filter layers which do not exhibit electrostatic properties perform both electrostatic filtration and mechanical filtration and are referred to as hybrid filters.

As shown in FIGS. 3, 6 and 7, the three filter layers 22, 23 and 24 are attached together along the entire peripheral marginal edge portions of the superposed middle and inner filter layers 23, 24 except at the central upper portions of the middle and inner filter layers. As best seen in FIG. 7, the three filter layers 22, 23 and 24 are not attached at the central upper portions of the middle and inner filter layers 23, 24 thereby creating an opening 27 between the outer and middle filter layers 22, 23 and an opening 28 between the middle and inner filter layers 23, 24. The opening 27 opens into a compartment between the outer and middle filter layers 22, 23 which are attached together along their peripheral margins except at the opening 27. Similarly, the opening 28 opens into a compartment between the middle and inner filter layers 23, 24 which are attached together along their peripheral margins except at the opening 28.

The two compartments between the three filter layers 22, 23 and 24 are provided for insertion therein of additional filter layers to enhance or modify the filtering properties of the nose-and-mouth filter portion 20. The insertion and removal of the middle layers may easily be done when the head garment is not being worn and is partly everted to expose the compartment openings 27, 28. The outer filter layer 22 constitutes part of the head garment fabric 2 (in this embodiment, a blend of spandex and polyester or nylon), and the middle and inner filter layers 23, 24 may also be comprised of the fabric 2 or other filter fabric materials. The additional filter layers may be comprised of any of the filter fabric layers described above or any other suitable filter material. The added filter layers may be of the same material as one or more of the filter layers 22, 23 and 24 or may be comprised of different materials.

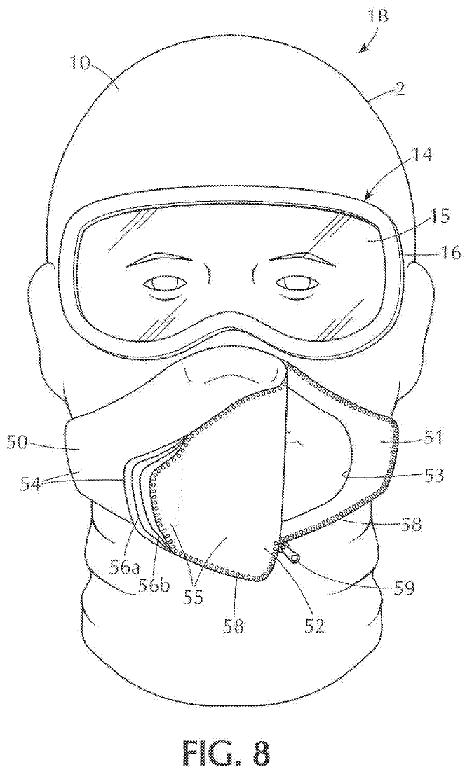

FIG. 8 illustrates a further embodiment of a head garment 1B worn by a wearer. The head garment 1B is the same as the head garment 1A illustrated in FIG. 3 except for the nose-and-mouth filter portion. In the FIG. 8 embodiment, a nose-and-mouth filter portion 50 (sometimes referred to hereinafter as filter portion 50) is removably attached to the head garment 1B. The filter portion 50 comprises a fixed part 51 fixed by stitching or other suitable fixation to the head garment 1B, and a removable filter part configured as a replaceable filter pack 52 removably attached to the fixed part 51.

The fixed part 51 may be made of fabric the same as or different from the fabric 2 and has an opening 53 that exposes the mouth and lower part of the nose of the wearer. Mating zipper tapes 58, 58 are attached to the respective marginal edge portions of the fixed part 51 and the removable filter pack 52. A zipper tab 59 is slidably attached to an end of the zipper tape 58 which is attached to the fixed part 51 for engagement with an end of the zipper tape 58 attached to the removable filter pack 52. In the disclosed embodiment, both ends of the zipper tape 58 attached to the fixed part 51 are attached at the left end of the fixed part as viewed in the drawing. The zipper tab 59 is pulled in a counterclockwise direction to engage or link the teeth of the zipper tapes 58, 58 and attach the removable filter pack 52 to the fixed part 51 and pulled in a clockwise direction to disengage or unlink the teeth of the zipper tapes and detach the removable filter pack 52 from the fixed part 51.

In FIG. 8, the removable filter pack 52 is partially cut and folded back to illustrate the multiple filter layers. The removable filter pack 52 has a multi-layer construction comprised of an outer filter layer 54 which defines the front surface of the filter pack 52, an inner filter layer 55 which defines the rear surface of the filter pack, and middle filter layers 56a, 56b interposed between the outer and inner filter layers 54, 55. The filter layers 54, 55, 56a, 56b may be made of the same or different materials. The number of middle filter layers is not limited to two, and one, three or more middle filter layers may be used. By way of example, the outer filter layer 54 and one or more other filter layers may be made of the fabric 2 and one of the middle layers 56a, 56b made of a material having electrostatic properties.

By making the filter pack 52 removable from the head garment 18, it is possible to easily remove and exchange the filter pack with a replacement filter pack, such as one having different filter properties, without the wearer having to remove the head garment. This is advantageous not only for children but also for older wearers. As known in the art, different filter layer materials generally have different filtering properties depending on the type and size of the airborne particles. An assortment of filter packs having different filter layer materials and different filter layer combinations can be made for use with the head garment 1B. This enables the wearer to select the filter pack best suited for the wearer's needs. For example, some filter packs may have filter layers selected to prevent airborne contaminants and other airborne substances such as smoke, dust, smog, pollen and mold from entering the wearer's respiratory system and other filter packs may have filter layers selected to protect other persons or things from being exposed to airborne pathogens such as viruses, bacteria and other microorganisms exhaled by the wearer. Also, a filter pack 52 which becomes compromised or which cannot be timely washed or sterilized/sanitized may be easily and quickly replaced by a new one without the wearer having to remove the head garment.

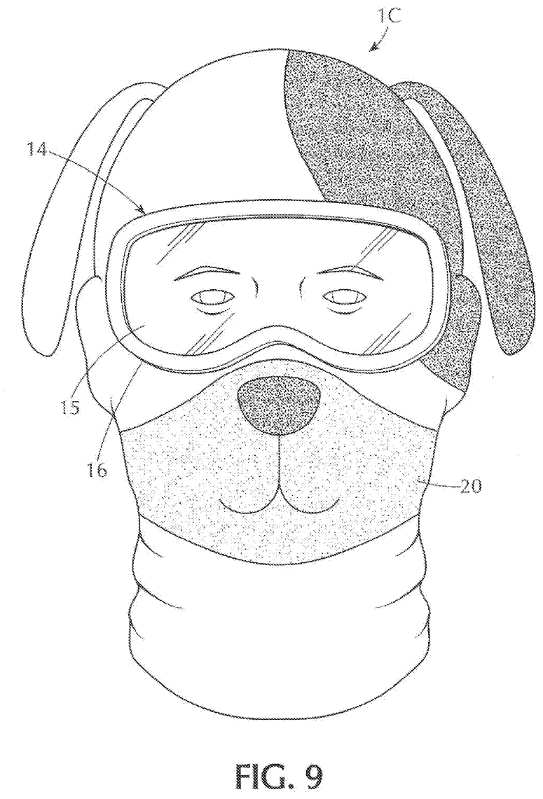

FIG. 9 illustrates another embodiment of a head garment 1C worn by a wearer. This embodiment is similar to the FIG. 3 embodiment, the difference being that the head garment 1C is configured as a fictious character, in this case a fanciful dog head. The head garment may be configured as the head of any desired fictious character, such as the head of an animal or superhero, which makes the head garment particularly attractive to children and encourages them to wear the head garment.

Various aspects of the present disclosure have been described with reference to different embodiments and examples which illustrate exemplary ways of implementing the various aspects. It is understood that one or more aspects of one head garment may be used with other head garments, and that different aspects of the different head garments may be substituted for corresponding aspects of other head garments. For example, the head garment embodiments illustrated in FIGS. 1-2 and in FIG. 9 may be equipped with any one of the protective eye shields illustrated in FIGS. 3-5. The multi-layer filter portion embodiment illustrated in FIG. 8 may be substituted for the multi-layer filter portion embodiments illustrated in FIGS. 1-3 and 6-7 and in FIG. 9.

Various changes and modifications to the embodiments and examples described above will be readily apparent to persons skilled in the art. The present invention encompasses all such changes and modifications that fall within the scope and spirit of the appended claims.

What is claimed is:

1. A head garment made of elastically stretchable fabric configured to fit over and surround the head of a wearer and conform substantially to the wearer's head, the head garment having an eye opening configured and positioned to allow the wearer to see therethrough, and a multi-layer filter portion configured to cover the wearer's mouth and at least the lower part of the wearer's nose, the multi-layer filter portion comprising at least three filter layers disposed one over the other, the at least three filter layers comprising an inner filter layer and a middle filter layer both attached to the backside of the head garment fabric which constitutes an outer filter layer.

2. The head garment according to claim 1; further including a protective eye shield completely covering the eye opening, the protective eye shield having transparent and scratch-resistant properties.

3. The head garment according to claim 2; wherein the protective eye shield is detachably attached to the head garment.

4. The head garment according to claim 2; wherein the protective eye shield comprises a transparent member fixed to a frame which is detachably attached to the head garment by fasteners, the fasteners having first mating parts fixed to the head garment fabric in spaced relation around a marginal edge portion of the eye opening, and second mating parts fixed in spaced relation to the underside of the frame and positioned to releasably engage with corresponding first mating parts when the protective eye shield is aligned with the eye opening.

5. The head garment according to claim 4; wherein the first and second mating parts comprise magnets, snaps or a combination thereof.

6. The head garment according to claim 2; wherein the protective eye shield has ultraviolet (UV) light-blocking properties.

7. The head garment according to claim 1; wherein the outer, middle and inner filter layers are attached together along the peripheral marginal edge portions of the middle and inner filter layers except along the central upper portions of the middle and inner filter layers thereby creating an opening between the outer and middle filter layers and between the middle and inner filter layers, the openings opening into two compartments, one between the outer and middle filter layers and the other between the middle and inner filter layers, the compartments being dimensioned to accommodate one or more additional filter layers inserted through the openings into the compartments to modify the filtering properties of the multi-layer filter portion.

8. The head garment according to claim 7; wherein the middle and inner filter layers are attached by stitching to the backside of the head garment fabric which constitutes the outer filter layer.

9. The head garment according to claim 8; wherein the middle and inner filter layers comprise fabric layers.

10. The head garment according to claim 9; wherein the outer, middle and inner filter layers are comprised of the same fabric.

11. The head garment according to claim 9; wherein two of the outer, middle and inner filter layers are comprised of the same fabric and the third filter layer is comprised of a fabric different from that of the other two filter layers.

12. The head garment according to claim 7; further including a protective eye shield completely covering the eye opening, the protective eye shield having transparent, scratch-resistant and ultraviolet (UV) light-blocking properties.

13. The head garment according to claim 1; wherein the multi-layer filter portion is removably attached to the head garment.

14. The head garment according to claim 1; wherein the head garment is configured as the head of a fictitious character.

15. A head garment made of elastically stretchable fabric configured to fit over and surround the head of a wearer and conform substantially to the wearer's head, the head garment having an eye opening configured and positioned to allow the wearer to see therethrough, and a multi-layer filter portion configured to cover the wearer's mouth and at least the lower part of the wearer's nose, the multi-layer filter portion comprising a fixed part fixed to the head garment fabric and having an opening configured to expose the wearer's mouth and at least the lower part of the wearer's nose, and a removable filter part configured as a replaceable filter pack removably attached to the fixed part to cover the opening, the filter pack comprising multiple filter layers disposed one over the other, the multiple filter layers comprising an outer filter layer which constitutes a front surface of the filter pack, an inner filter layer which constitutes a rear surface of the filter pack, and one or more middle filter layers superposed between the outer and inner filter layers.

16. The head garment according to claim 15; wherein the outer filter layer is made of the head garment fabric.

17. The head garment according to claim 15; wherein the filter pack has one or more additional filter layers interposed between the outer and inner filter layers.

18. The head garment according to claim 17; wherein the filter properties of some of the filter layers are different from the filter properties of other filter layers.

19. The head garment according to claim 15; further including a protective eye shield completely covering the eye opening, the protective eye shield having transparent and scratch-resistant properties.

20. The head garment according to claim 15; wherein the head garment is configured as the head of a fictitious character.
